(19) United States
(12) Patent Application Publication
(10) Pub. No.: US 2018/0158559 A1
(43) Pub. Date: Jun. 7, 2018

(54) METHOD AND SYSTEM FOR PRODUCING GALLIUM-68 RADIOISOTOPE BY SOLID TARGETING IN A CYCLOTRON

(71) Applicant: NCM USA BRONX LLC, Bronx, NY (US)

(72) Inventors: Ali A. Abbasi, New York, NY (US); Balu Easwaramoorthy, Dumont, NJ (US)

(21) Appl. No.: 15/579,823

(22) PCT Filed: Jun. 6, 2016
(86) PCT No.: PCT/US16/35971

§ 371 Date: Dec. 5, 2017

Related U.S. Application Data
(60) Provisional application No. 62/171,453, filed on Jun. 5, 2015.

(51) Int. Cl. G21G 1/10; G21G 1/00; A61K 51/08

(52) U.S. Cl. CPC G21G 1/10; G21G 1/001; G21Y 2004/10; G21G 2001/0021; A61K 51/08

(57) ABSTRACT

In a system and a method for making carrier-free radioactive isotopic Gallium-68, stable enriched Zinc-68 is formed into a solid target of very high purity. The solid target of enriched Zinc-68 is exposed to a proton beam provided by irradiation in a cyclotron to change the enriched Zinc-68 into Gallium-68. After irradiation, the solid target contains high concentrations of Gallium-68 with only trace amounts of enriched Zinc-68 and isotopic Gallium-67. Gallium-68 is then further purified to remove the impurities resulting in a Gallium-68 composition with high purity and specific activity and without Germanium-68, Also provided are radiopharmaceutical agents that are labeled with the Gallium-68 compositions made by solid targeting in a cyclotron.

### CROSS-REFERENCE TO RELATED APPLICATIONS

This application claims the benefit of priority of U.S. Provisional Patent Application No. 62/171,453, filed Jun. 5, 2015, which is incorporated by reference in its entirety.

### FIELD OF THE INVENTION

The present invention relates to the generation of Gallium-68 (Ga-68) from enriched Zinc-68, and particularly a method and system for generating Ga-68 using solid targeting of enriched Zinc-68 using a cyclotron.

### BACKGROUND

Gallium-68 (Ga-68) is a positron emitting radioactive isotope (E_{βmax}=1.8 MeV, β⁺=89%) with a short half-life (t_{1/2}=67.7 minutes). Ga-68 is generally produced using a generator. Generator based methods use Germanium-68 (Ge-68) as the parent isotope for Ga-68 (U.S. Pat. No. 4,264,468). Ga-68 has important uses including for diagnostic positron emission tomography (PET) scans of various rapidly changing processes and targets. PET is a non-invasive medical imaging technology that is useful for generating high-resolution images that are important for medical diagnostics in oncology, cardiology, and neurology. When used in PET scans, Ga-68 is attached to another molecule to form a radioactive tracer. Ga-68 is also useful as a radiopharmaceutical when attached to a pharmaceutical moiety.

The short half-life of Ga-68 and long half-life of Ge-68 present two distinct problems. First, due to the short half-life of Ga-68, it is critical that Ga-68 be produced with high yield and purity in order to compensate for the short half-life and to minimize the need for extensive time consuming purification steps. In order to minimize the amount of decay, Ga-68 must also be produced and then converted into a radioactive tracer shortly before use and near the location at which it will be used. This requires hospitals to maintain on-site generators and facilities for purifying and manipulating the Ga-68. Second, due to the much longer half-life of Ge-68 (271 days), Ge-68 has unwelcome side effects in the body. This results in the need for complete separation of Ga-68 from Ge-68 prior to use. Previously described methods of separation are either not able to provide complete separation (U.S. Pat. No. 4,248,730) or not able to provide sufficiently high yields (U.S. 2013/0055855).

One alternative to Ge-68/Ga-68 generators includes use of a cyclotron. In cyclotron based methods, a liquid or solid target is irradiated with a proton beam.

### SUMMARY

In one cyclotron based method, a solid target of Zinc-68 electrodeposited onto a copper backing is used. However due to the presence of the copper backing, lengthy separation steps are required, limiting the yield of the short lived Ga-68 isotope. Liquid targets of Zinc nitrate solutions do not require these separation steps but previously described attempts have failed to produce Ga-68 with sufficient yield or specific activity. Based on the increasing applicability of Ga-68 labelled radiopharmaceuticals, there is a need for an alternative approach and method for producing Ga-68 with high purity, high yield, and specific activity.

The present invention provides methods for making and purifying a carrier free radioactive isotope Gallium-68 (Ga-68) and radiolabeled carrier molecules therefrom.

The present invention also provides a system for producing carrier free radioactive isotope Ga-68 and radiolabeled carrier molecules therefrom.

The present invention also provides compositions including a carrier-free radioactive isotope Ga-68, where the composition is free of Germanium-68.

The present invention also provides compositions comprising one or more carrier molecule, where the one or more carrier molecule is radiolabeled with the Ga-86 according to the above compositions.

Example embodiments of the present invention provide methods for making a carrier free radioactive isotope Ga-68 by irradiating a solid target of substantially pure enriched Zinc-68 with a proton beam provided by a cyclotron to produce Ga-68. Preferably, the solid target is 99% enriched Zinc 68. Preferably, the solid target is a foil. Preferably, the solid target is about 0.05 to about 1.0 mm thick. Preferably, the solid target has a molar content of enriched Zinc-68 that is about 0.01 to about 1.0 mmol. Preferably, the solid target is irradiated for about 1 to about 2 hours. Preferably, the proton beam provided by the cyclotron has an intensity of about 10 to about 16 MeV. Preferably, the proton beam is directed at the solid target with an angle of incidence of about 10 to about 90 degrees.

Example embodiments of the present invention also provide methods of purifying the carrier free radioactive isotope Ga-68 made according to any one of the above methods and further including purifying the produced Ga-68 by dissolving the irradiated solid target in a dissolving acid, isolating Ga-68 from the dissolved solid target, washing with at least one washing solution, and recovering purified Ga-68. Preferably, the dissolving acid is a strong acid and more preferably the dissolving acid has a normality of about 8 to about 12 N. Preferably, the Ga-68 is isolated using an ion exchange column and more preferably, the ion exchange column contains an anion exchange resin. Preferably, the ion exchange column containing isolated Ga-68 is washed with at least one washing solution more than once. Preferably, the washing solution is water or an aqueous solution of hydrobromic acid and acetone, more preferably the washing solution is 0.5M hydrobromic acid in 80% acetone or an equivalent thereof, and most preferably the ion exchange column containing isolated Ga-68 is washed at least twice where one of the at least one washing solution is water and one of the at least one washing solution is 0.5M hydrobromic acid in 80% acetone or an equivalent thereof. Preferably, the Ga-68 is recovered from the ion exchange column using an elution solution and, more preferably, the elution solution is about 0.05 to about 3.0 M hydrochloric acid or equivalents thereof. Preferably, the method has a production yield of Ga-68 that is at least about 1 Gbq/μAh to about 5 Gbq/μAh.

Example embodiments of the present invention also provide a system for producing carrier free radioactive isotope Ga-68, the system including a solid target of substantially pure enriched Zinc-68; a cyclotron, where the solid target is irradiated using the cyclotron according to any one of the above methods of making; and an ion exchange column, where the irradiated solid target is purified according to the steps of any one of the above mentioned methods of purifying.

Example embodiments of the present invention also provide compositions made according to any one of the above methods including a carrier-free radioactive isotope Ga-68, where the composition is free of Ge-68. Preferably, the composition is at least about 99% Ga-68. Preferably, the composition is less than about 0.1% of Gallium-67. Preferably, the composition has a specific activity of at least about 3.0 Gbq/μg to about 8.5 Gbq/μg.

Example embodiments of the present invention also provide carrier molecules radiolabeled with the Ga-68 according to the above compositions. Preferably, the carrier molecule is a drug, protein, antibody, antibody fragment, peptide, peptide fragment, or particle. More preferably, the carrier molecule is selected from the group consisting of: prostate-specific membrane antigen (PSMA), 1,4,7-triazacyclo-NN,N′N″-triacetic acid (NOTA); 1,4,7,10-tetraazacyclododecane-1,4,7,10-tetraacetic acid (DOTA); diethylene triamine pentaacetic acid (DTPA); 1,4,7-triazacyclononane-1,4,7-triacetic acid (NOTA); Desferrioxamine, DOTA-Tyr(3)-octreotide (DOTATOC); DOTA-Tyr(3)-Tyr(8)-octreotide (DOTATATE); DOTA-1-naphtyl-alanine (DOTANOC); DOTA-benzothienyl-alanine (DOTA-BOC); DOTA-bombesin; DOTA-arginine-glycine-aspartic acid-bombesin (DOTA-RGD-bombesin); 1,4,7-triazacyclononane-1,4,7-triacetic acid-RGD (NOTA-RGD); 3,6,9,15-tetraazabicyclo[9.3.1]pentadeca-1(15),11,13-triene-3,6,9-triacetic acid-RGD (PCTA-RGD); DOTA-albumin; DOTA-human epidermal growth factor; 1,4,7-triazacyclononane-1-[methyl(2-carboxyethyl)phosphinic acid]-4,7-bis[methyl(2-hydroxymethyl)phosphinic acid-integrin alpha(IIb)beta(3)-specific cyclic hexapeptide (NOPO-RGDfK); 1,4,7-triazacyclononane-1,4-bis(acetic acid)-7-(2-glutaric acid) (NODAGA); NOPO-NaI(3)-octreotide conjugate (NOPO-NOC); and 1,4,7-triazacyclononane-1,4,7-tris[(2-carboxyethyl)methylenephosphinic acid] (TRAP(RGD)₃). Most preferably, the carrier molecule is PSMA. Preferably, the carrier molecule targets a human tissue. More preferably, the human tissue is selected from the group consisting of: thyroid, brain, gastrointestinal, pancreas, spleen, kidney, neuroendocrine tumors, renal cell carcinoma, small cell lung cancer, breast cancer, prostate cancer, and malignant lymphoma.

These and other embodiments of the invention will be better appreciated and understood when considered in conjunction with the following description and the accompanying drawings. However, the following description, while indicating various embodiments of the invention and numerous specific details thereof, is given by way of illustration and not of limitation. Many substitutions, modifications, additions and/or rearrangements can be made within the scope of the invention without departing from the spirit thereof, and the invention includes all such substitutions, modifications, additions and/or rearrangements.

The drawings accompanying and forming part of this specification are included to depict certain aspects of the invention. A clearer conception of the invention and of the components and operation of systems provided with the invention will become more readily apparent by referring to the exemplary, and therefore non-limiting, embodiments illustrated in the drawings, wherein like reference numerals (if they occur in more than one view) designate the same or similar elements. The invention may be better understood by reference to one or more of these drawings in combination with the description presented herein. The features illustrated in the drawings are not necessarily drawn to scale.

### BRIEF DESCRIPTION OF THE DRAWINGS

FIG. 1 shows a spectrogram from HPGe analysis for radionuclide purity and identity of Ga-68 according to an example embodiment of the present invention.

FIG. 2 is a chromatograph from HPLC analysis of Ga-68 according to an example embodiment of the present invention after purification by cation exchange column.

FIG. 3 is a chromatograph from HPLC analysis of PSMA radiolabeled with G-68 according to an example embodiment of the present invention.

FIG. 4 is a chromatograph from HPLC analysis of PSMA radiolabeled with generator produced Ga-68 which is shown for comparative purposes.

FIG. 5 is a chromatograph from HPLC analysis of purified PSMA radiolabeled with Ga-68 according to an example embodiment of the present invention.

FIG. 6 shows a PET image of an LNCaP xenograft bearing mouse injected with PSMA radiolabeled with Ga-68 according to an example embodiment of the present invention.

### DETAILED DESCRIPTION

The inventors have discovered that the radioactive isotope Gallium-68 (Ga-68) can be prepared utilizing the Zinc-68(p,n), Ga-68 nuclear reaction in cyclotrons. In particular the Ga-68 prepared according to the invention has high-purity/high-radioactivity concentration and allowing for production at lower cost than conventional methods of Ga-68 production. Advantageously, there are no Germanium-68 (Ge-68) impurities in the Ga-68 preparations made utilizing the Zinc-68(p,n), Ga-68 nuclear reaction in medium to low-energy cyclotrons resulting in a savings of both time and costs relating to the separation of Ge-68 impurities from the Ga-68.

The present invention provides for carrier-free radioactive isotope Ga-68 having high-purity/high-radioactivity concentrations and that is free of Ge-68 impurities, and carrier molecules radiolabeled with Ga-68 thereof. In this manner, unlike Ga-68 produced using generators, there is no risk of carrier molecules being labeled with or contaminated by Ge-68 when labeling using the Ga-68 of the present invention.

The present invention also provides a method for producing the radioactive isotope Ga-68 from a solid target of enriched Zinc-68 using a cyclotron and making radiolabeled carrier molecules therefrom.

The present invention also provides a system for producing the radioactive isotope Ga-68 and radiolabeled carrier molecules therefrom using a solid target of enriched Zinc-68 in a cyclotron, where the Ga-68 has a high-purity/high-radioactivity concentration that is free of Ge-68 impurities.

The term “carrier molecule” as used herein means a drug, protein, antibody, antibody fragment, peptide, peptide fragment, or particle, which when introduced into the body by injection, swallowing, or inhalation accumulates in one or more organs or tissues of interest. The organ(s) or tissue(s) where accumulation occurs is said to be the target organ(s) or target tissue(s) of the carrier molecule, Examples of carrier molecules include but are not limited to: prostate-specific membrane antigen (PSMA); 1,4,7-triazacyclo-NN,N′N″-triacetic acid (NOTA); 1,4,7,10-tetraazacyclododecane-1,4,7,10-tetraacetic acid (DOTA); diethylene triamine pentaacetic acid (DTPA); 1,4,7-triazacyclononane-1,4,7-triacetic acid (NOTA); Desferrioxamine, DOTA-Tyr(3)-octreotide (DOTATOC), DOTA-Tyr(3)-Tyr(8)-octreotide (DOTATATE); DOTA-1-naphtyl-alanine (DOTANOC); DOTA-benzothienyl-alanine DOTA-BOC); DOTA-bombesin; DOTA-arginine-glycine-aspartic acid-bombesin (DOTA-RGD-bombesin); 1,4,7-triazacyclononane-1,4,7-triacetic acid-RGD (NOTA-RGD); 3,6,9,15-tetraazabicyclo[9.3.1]pentadeca-1(15),11,13-triene-3,6,9-triacetic acid-RGD (PCTA-RGD); DOTA-albumin; DOTA-human epidermal growth factor; 1,4,7-triazacyclononane-1-methyl[(2-carboxyethyl)phospinic acid]-4,7-bis[methyl(2-hydroxymethyl)phosphinic acid-integrin alpha(IIb)beta(3)-specific cyclic hexapeptide (NOPO-RGDfK); 1,4,7-triazacyclononane-1,4-bis(acetic acid)-7-(2-glutaric acid) (NODAGA); NOPO-NaI(3)-octreotide conjugate (NOPO-NOC); and 1,4,7-triazacyclononane-1,4,7-tris[(2-carboxyethyl)methylenephosphinic acid] (TRAP(RGD)₃). Examples of targets include but are not limited to: thyroid, brain, gastrointestinal, pancreas, spleen, kidney, neuroendocrine tumors, renal cell carcinoma, small cell lung cancer, breast cancer, prostate cancer, and malignant lymphoma.

The term “strong acid” as used herein means an acid with a pKa<−1.74 that ionizes completely in an aqueous solution by losing One proton according to the equation: HA(aq)→H⁺(aq)+A⁻(aq). Examples of strong acids include hut are not limited to: perchloric acid, hydroiodic acid, hydrobromic acid, hydrochloric acid, sulfuric acid, p-toluenesulfonic acid, methanesulfonic acid, fluoroantimonid acid, magic acid, carborane superacid, fluorosulfuic acid, and trifilic acid.

Commercially available proton beam cyclotrons are known in the art, e.g., the PETtrace 880 cyclotron manufactured by GE Healthcare (Husbyborg, Sweden), which is not a variable MeV instrument. There are examples of commercially available cyclotron instruments allowing for variable energy, for example the ACS PET trace system, but the energy cannot be reduced below 14 meV.

Commercially available cyclotrons can also be modified to change the energy level of the proton beams. For example, in an example embodiment, the PETtrace 880 is modified to produce Ga-68 by irradiation of enriched Zinc-68 with a proton beam to cause the Zinc-68(p,n), Ga-68 reaction. In an example embodiment, the cyclotron is modified by introducing an energy attenuation disc in the beam path in order to reduce the MeV of the GE PETtrace 880, where the energy attenuation disc is made of aluminum foil and cooled by a helium flow to minimize the production of byproduct and other impurities. In an example embodiment, the energy attenuation disc is placed between the vacuum foil and the target material. Using the energy attenuation disc, the energy of the proton beam is adjusted from the default setting of 16.5 MeV to about 11 to 12 MeV. In an example embodiment, the thickness of the energy attenuation disc used to reduce the energy to 11 to 12 MeV is about 0.6 to 0.9 mm. The energy of the beam can also be adjusted to other energy levels by adjusting the thickness of the energy attenuation disc. The present invention is not limited to only the PETtrace 880, but can be equally implemented using other cyclotron models provided the cyclotron can produce a beam energy in the range of 10 MeV to 16 MeV and preferably can be adjusted or modified to produce a beam having an energy level of 11 to 12 MeV.

Preferably, the energy level of the beam is set to 10 MeV to 16 MeV. Even more preferably, the energy level of the beam is adjustable within the range of 10 MeV to 16 MeV. An energy level lower than 11 MeV results in no measurable amount of Ga-67, but a low-yield of Ga-68. Energy levels exceeding 12 MeV increases Ga-68 yield, but also increases the amount of resulting Ga-67 impurity. Therefore, the energy level is preferably 11<E<12. Most preferred, the energy level is 11.5 MeV. Preferably, a beam current of at least 30-60 μA is used. One method for obtaining higher Ga-68 yields while minimizing the level of impurities is to increase the current (μA) used on the target material while maintaining the optimal energy level of about 11.5 MeV. Utilizing higher currents produces more heat, but this is addressed by providing additional cooling. Using solid targets as opposed to liquid targets allows for higher beam currents to be applied. Liquid targets have the disadvantage of boiling and evaporating when high beam currents are used due to excessive heating. Solid targets do not suffer from this boil off limitation. Furthermore, cooling methods may be more readily applied to solid targets than liquid targets while maintaining beam exposure. Finally, solid targets allow for higher concentration of the target material and therefore result in higher concentrations of the desired radioactive isotope.

In an example embodiment, enriched Zinc-68 (>99% purity) is the starting material and is used as a solid target in the cyclotron. Enriched Zinc-68 is prepared as a neat foil with 250-800 mg enriched Zinc-68 mass content. The thickness and size of the target made from the foil is customized to fit the solid target support of the cyclotron used but can be varied depending on the model of the cyclotron. In particular, in an example case, the foil was cut to a size of 20×100 mm and inserted into a target holding device made of aluminum.

In the cyclotron, the Zinc-68 foil target is separated from the cyclotron tank vacuum by two windows. The first window is an energy attenuation disc made from aluminum foil. The second window is a 0.025 mm thick high-strength non-magnetic alloy foil, for example Havar foil (Goodfellow, Coraopolis, Pa.). Both windows are oriented at a 90° angle to the proton beam. In the example case, helium flow was directed through the space between the target foil and the Havar foil to dissipate heat induced in the foils by the beam as well as through the space surrounding the energy attenuation disc. The enriched Zinc-68 foil target was placed approximately 10 cm behind the second window at a 10° angle to the beam in order to increase effective thickness of the target and improve heat dissipation. The enriched Zinc-68 foil target was supported by a water-cooled aluminum plate. A volume between the second window and the target was sealed and filled with inert gas (helium) to prevent oxidation.

In an example, beam current of 30-60 μA is maintained for 1-2.5 hours to produce the required amount of Ga-68, and, after the irradiation, the foil target is left on the cyclotron for a predetermined amount of time so that any short-lived product could decay. For example, in an example case, the irradiated foil target was allowed to cool down for 5 to 15 minutes to allow short-lived byproducts to decay. The irradiated foil target was then removed from the holder and transported into a processing hot cell. The cyclotron and processing hot cell can be configured to automate transport of the irradiated foil target, or the target can be manually moved, from the cyclotron vault to the processing hot cell. An automated transportation system has the added advantage of limiting radiation exposure (approximately 2-3 Ci radioactivity) experienced while transporting target materials to the designated processing hot cell. The target transport system also allows for time savings which are significant for short-lived materials due to radioactive decay. The transport system further prevents any possible contamination to the target material during transportation that could affect the purity or purification process, resulting in lower than usual expected yield.

In an example embodiment, separation of Ga-68 from Zinc-68 is accomplished using a cation-exchange method. For example, in an example case, cation exchange column containing 0.5-2.0 g of AG-50W-X8 resin was used for Ga-68 trapping. The cation exchange column was conditioned with water followed by air. The irradiated target was dissolved in about 10-12 N HCl. The dissolved target solution was passed through a cation exchange column. The cation exchange column effectively trapped both the Ga-68 and Zinc-68. Trapping was 100% and no residual radioactivity was observed in the volume that passed through the cation column. After trapping, the column was washed with 5.0 mL of chelexed water to remove any metal contamination and any short-lived isotopes. Zinc-68 was then eluted from the column using 30 mL of 0.5 N HBr in 80% acetone solution and collected in a separate recovery vial, followed by a 3 mL water rinse to remove any remaining HBr-acetone. Finally, Ga-68 was eluted with 3 N HCl (2-3 mL) to a product vial. Following elution, the eluent and cation exchange column was measured for radioactivity using a calibrated dose calibrator to confirm complete elution. The separation procedure can be conducted manually or automated to ensure consistent production and lower exposure to radioactivity.

### EXAMPLES

### Example 1

### Ga-68 Produced Using Solid Targetry of Enriched Zinc-68 in a Cyclotron

A PETtrace 880 cyclotron was modified using a 0.6 mm aluminum foil energy attenuation disc in order to reduce the MeV of the GE PETtrace 880 from 16.5 MeV to about 11-12 MeV. The energy attenuation disc was placed in the proton beam path, after the vacuum foil of the target, and before the target material. A beam current of 55 μA was used to irradiate the target for about 1.2 hours. The solid target was made from enriched Zinc-68 (>99% purity). Enriched Zinc-68 was prepared as a neat foil with 250-800 mg enriched Zinc-68 mass content. The thickness and size of the target made from the foil was customized to fit the solid target support of the cyclotron. In particular, the foil was cut to 20×100 mm in size and inserted into a target holding device made of aluminum.

After the irradiation, the foil target was left on the cyclotron and allowed to cool down for 5 to 15 minutes so that any short-lived product could decay. The irradiated foil target was then manually removed from the holder and transported into the processing hot cell.

Separation of Ga-68 from Zinc-68 was accomplished using a cation-exchange method. A cation exchange column containing 0.5-2.0 g of AG-50W-X8 resin was used for Ga-68 trapping. The cation exchange column was conditioned by flowing through water followed by air. The irradiated target was dissolved in about 10-12 N HCl. The dissolved target solution was passed through a cation exchange column. The cation exchange column effectively trapped 100% of both the Ga-68 and Zinc-68. Complete trapping was confirmed in that no residual radioactivity was observed in the volume that passed through the cation column. After trapping, the column as washed with 5.0 mL of chelexed water to remove any metal contamination and any short-lived isotopes. Zinc-68 was then eluted from the column using 30 mL of 0.5 N HBr in 80% acetone solution and collected in a separate recovery vial, followed by a 3 mL water rinse to remove any remaining HBr-acetone. Finally, Ga-68 was eluted with 3 N HCl (3 mL) to a product vial. The elution resulted in a total of about 1865 mCi of the radioisotope. Following elution, the eluent and cation exchange column was measured for radioactivity using a calibrated dose calibrator to confirm complete elution.

Samples prepared using the above methods were analyzed for production yield and specific activity. The activity of the obtained Ga-68 was 66.6 to 360.75 Gbq at the end of bombardment (EOB). Production yields of 68Ga and 67Ga were about 0.93 Gbq/μAh to about 5.2 Gbq/μAh and <3.7×10⁻⁵ Gbq/μAh, respectively, at the EOB. The nuclear reaction mechanism for 68Ga and 67Ga are interpreted to ⁶⁸Zn(p,n) ⁶⁸Ga and ⁶⁸Zn(p,2n) ⁶⁷Ga, respectively. The chemical separation yield was 90%, and the chemical separation time was less than 10 min.

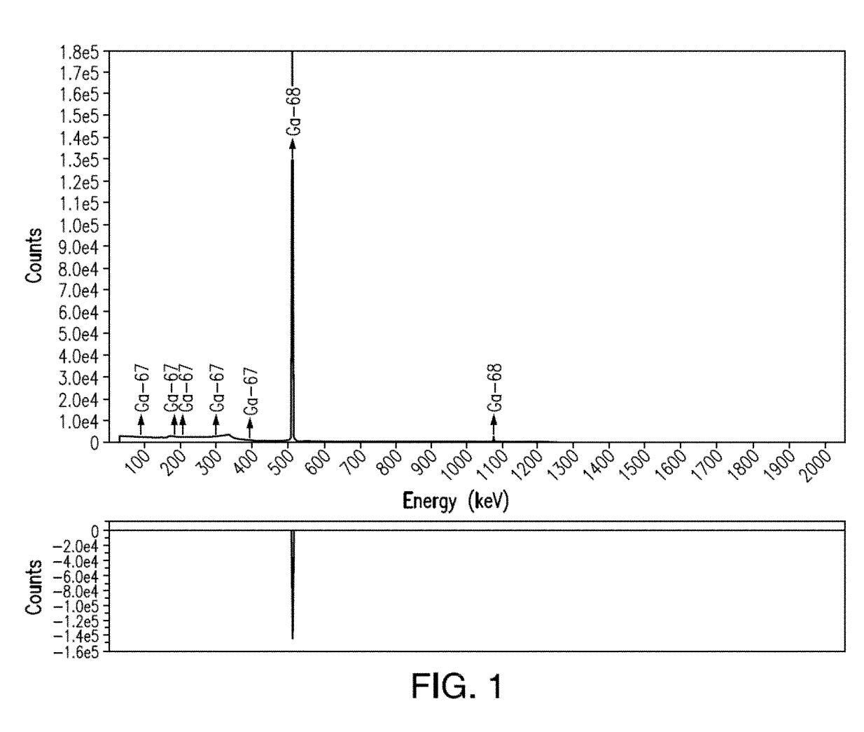

Samples were also analyzed for radionuclide purity and identity using an ORTEC GEM series high-purity Germanium (HPGe) coaxial detector system (model GEM20-70-SMP, CFG-SV-70). The purified sample of Ga-68 showed only two peaks at 511 keV and 1077 keV, both corresponding to Ga-68 (FIG. 1). The specific activity (Gbq/μg) of Ga-68 was measured by determining the total Ga metal present in the final product after purification. Ga-68 was measured as having a specific activity in the range of about 3.1 Gbq/μg to about 8.5 Gbq/μg (decay corrected to EOB). The other metal contaminants including Zn, Fe, and Ga were also analyzed using an ICP-mass spectrometer. The radionuclide purity of Ga-68 was >99.9%. The only impurity present was Ga-67 in very small quantities. Advantageously, no Germanium-68 is present.

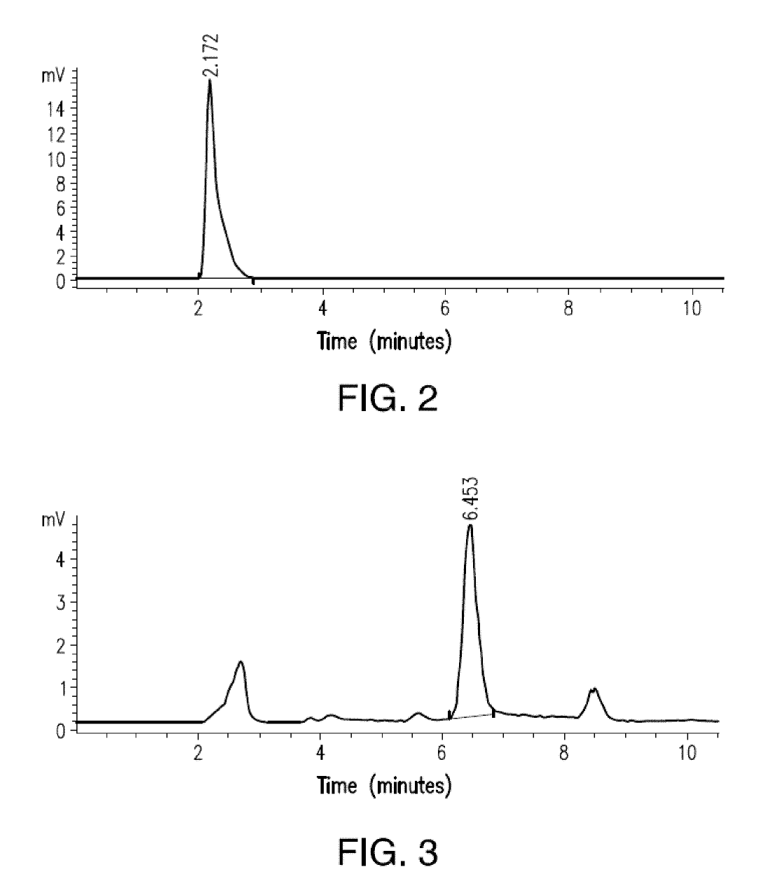

The purified Ga-68 was further characterized using 1100 series Agilent analytical HPLC equipped with UV and radiometric detectors. A reverse phase C18 Phenomenix (4×250 min) column was used. The mobile phase was 20% acetonitrile in 0.1% TFA and the flow rate was 0.7 mL/min. The purified sample showed a single peak at 2 minutes corresponding to Ga-68, indicating no other presence of radio-impurities (FIG. 2). Therefore, the purification step was able to remove the small amount of Ga-67 impurities present.

### Example 2

### PSMA Radiolabeled with Cyclotron Produced Ga-68

To confirm that the Ga-68 produced according to the above method is capable of radiolabeling carrier molecules in the same manner as Ga-68 produced according to other methods, the Ga-68 produced according to the above method was used to radiolabel DKFZ-GaPSMA-11 prostate-specific membrane antigen (PSMA) (ABX advanced biochemical compounds Germany). Purified Ga-68 was pH-adjusted with 10N NaOH and diluted to give a final concentration of 100 mCi/mL at pH 4.5. For PSMA labeling, the 20-50 μg PSMA (about 40 nanomoles) was added to the Ga-68 and heated at 37° C. for 9 minutes. The progress of the reaction was monitored using analytical HPLC. The PSMA radiolabeled with cyclotron produced. Ga-68 (cyclotron Ga-68-PSMA) was then analyzed using HPLC. The HPLC showed two peaks corresponding to carrier-free Ga-68 (at about 2 min) and Ga-68 labeled PSMA (at about 6.5 minutes). Satisfactory radiolabeling yield (90-95% of PSMA) was observed for the above conditions (FIG. 3). After labeling, 60 mCi (2.2 Gbq) of cyclotron Ga-68-PSMA was obtained in 10 mL 10% ethanol saline.

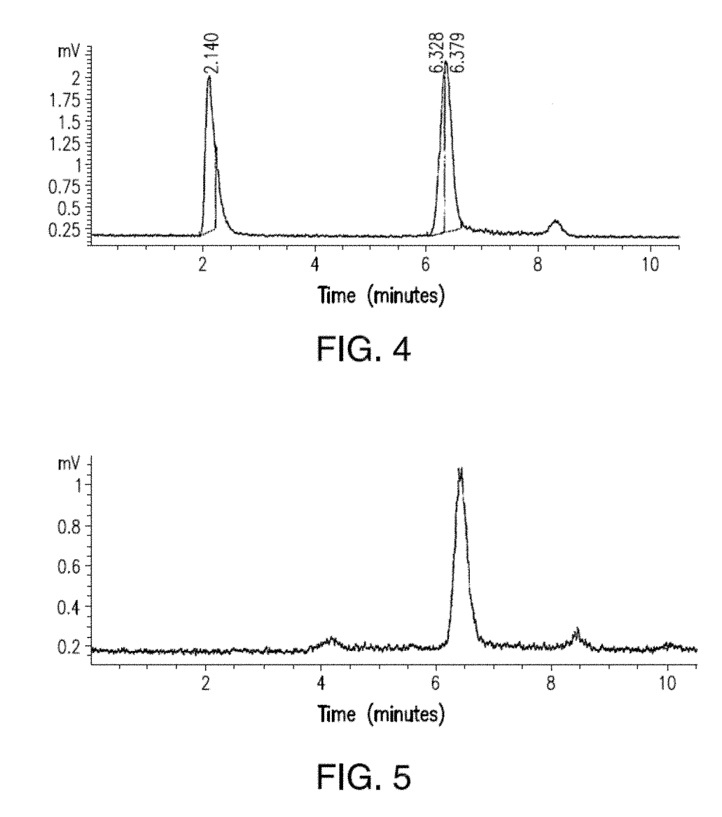

Radiolabeling of DKFZ-GaPSMA-11 was repeated using identical conditions except that generator produced Ga-68 was used (FIG. 4). The results were then compared to the DKFZ-GaPSMA-11 radiolabeled with cyclotron produced Ga-68. The results confirmed that cyclotron produced Ga-68 was able to provide radiolabeled carrier molecules with identical radiochemical properties as those of generator produced Ga-68 but without the risk of contamination from Germanium-68 impurities present in generator produced Ga-68. Finally, the PSMA radiolabeled with cyclotron produced Ga-OS was purified using a SEP-PAK® C-18 Column Chromatography Cartridge (Flinn Scientific, Batavia, Ill.) to remove carrier-free Ga-68 (FIG. 5).

To confirm bioequivalence of the cyclotron Ga-68-PSMA, 250 μCi (9 Mbq) of the cyclotron Ga-68-PSMA was injected into androgen-sensitive human prostate adenocarcinoma cell (LNCaP) xenograft bearing mice. These xenograft hearing mice serve as models for prostate cancer tumors and were used to determine the ability of the cyclotron Ga-68-PSMA to detect prostate cancer tumors. Thirty minutes after injection the mice were imaged in a PET scanner. The PET images were analyzed to determine whether the cyclotron Ga-68-PSMA could be imaged and, if imaged, whether the cyclotron Ga-68-PSMA was able to target the LNCaP xenograft.

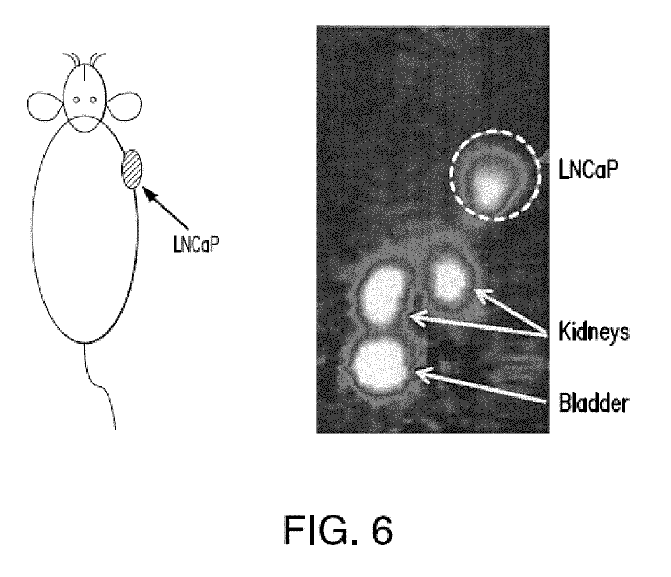

PET imaging confirmed that cyclotron Ga-68-PSMA was successfully imaged using the PET scanner and that the cyclotron Ga-68-PSMA targeted the LNCaP xenograft (FIG. 6). The urinary system (kidneys and bladder) was also visualized in the PET image as the cyclotron Ga-68-PSMA was filtered by the urine. The resulting images and targeting of cyclotron Ga-68-PSMA were as expected and confirmed bioequivalence of the cyclotron Ga-68-PSMA with PSMA radiolabeled with Ga-68 produced using other methods of production. Therefore, the cyclotron produced Ga-68 can be used as a direct substitute for Ga-68 produced using other methods, such as by generators, while providing the additional benefit of having no risk of contamination from Ge-68. This eliminates the need for additional purification steps aimed towards removing Ge-68, which increase the time spent on processing and the amount of decay experienced prior to use.

The above description is intended to be illustrative, and not restrictive. Those skilled in the art can appreciate from the foregoing description that the present invention may be implemented in a variety of forms, and that the various embodiments may be implemented alone or in combination. Therefore, while the embodiments of the present invention have been described in connection with particular examples thereof, the true scope of the embodiments and/or methods of the present invention should not be so limited since other modifications will become apparent to the skilled practitioner upon a study of the drawings, and specification.

1. A method of making carrier free radioactive isotope Gallium-68, the method comprising:
irradiating a solid target of substantially pure enriched Zinc-68 with a proton beam provided by a cyclotron to produce Gallium-68.

2. The method according to claim 1, wherein the solid target is 99% enriched Zinc-68.

3. The method according to claim 1, wherein the solid target is a foil.

4. The method according to claim 1, wherein the solid target is about 0.05 to about 1.0 mm thick.

5. The method according to claim 1, wherein the solid target has a molar content of enriched Zinc-68 that is about 0.01 to about 1.0 mmol.

6. The method according to claim 1, wherein the proton beam has an intensity of about 10 to about 16 MeV.

7. The method according to claim 1, wherein the solid target is irradiated for about 1 to about 2 hours.

8. The method according to claim 1, wherein the proton beam is directed at the solid target with an angle of incidence of about 10 to about 90 degrees.

9. The method of claim 1, further comprising:
dissolving the irradiated solid target in a dissolving acid;
isolating Gallium-68 from the dissolved solid target;
washing with at least one washing solution; and
recovering purified Gallium-68.

10. The method of claim 9, wherein the dissolving acid is a strong acid.

11. The method of claim 9, wherein the dissolving acid has a normality of about 8 to about 12 N.

12. The method of claim 9, wherein the Gallium-68 is isolated using an ion exchange column.

13. The method of claim 12, wherein the ion exchange column contains an anion exchange resin.

14. The method of claim 9, wherein the washing step is repeated more than once.

15. The method of claim 9, wherein the washing solution is an aqueous solution of hydrobromic acid and acetone.

16. The method of claim 9, wherein the washing solution is 0.5M hydrobromic acid in 80% acetone.

17. The method of claim 9, wherein the washing solution is water.

18. The method of claim 12, wherein the Gallium-68 is recovered from the ion exchange column using an elution solution.

19. The method of claim 18, wherein the elution solution is about 0.05 to about 3.0 M hydrochloric acid.

20. The method of claim 12, wherein a production yield of purified Gallium-68 of the method is at least about 1 Gbq/μAh to 5 Gbq/μAh.

21. A system for production of carrier free radioactive isotope Gallium-68 comprising:
a solid target of substantially pure enriched Zinc-68;
a cyclotron configured to irradiate wherein the solid target with a proton beam to produce Gallium-68; and
an ion exchange column configured to isolate the Gallium-68 from the solid target when the solid target is in a dissolved state, in a purification process that includes dissolving the irradiated solid target in a dissolving acid, performing the isolation of the Gallium-68 from the dissolved solid target, washing the ion exchange column with at least one washing solution, and recovering purified Gallium-68.

22. A composition comprising a carrier-free radioactive isotope Gallium-68, wherein the composition is at least about 99% Gallium-68, the composition is less than about 0.1% of Gallium-67, and the composition is free of Germanium-68.

23. The composition of claim 22, wherein the Gallium-68 has a specific activity of at least about 3 Gbq/μg to about 8.5 Gbq/μg.

24. The composition of claim 22, further comprising a carrier molecule, wherein the carrier molecule is radiolabeled with the Gallium-68.

25. The composition of claim 24, wherein the carrier molecule is selected from the following: prostate-specific membrane antigen (PSMA); 1,4,7-triazacyclo-NN,N′N″-triacetic acid (NOTA); 1,4,7,10-tetraazacyclododecane-1,4,7,10-tetraacetic acid (DOTA); diethylene triamine pentaacetic acid (DTPA); 1,4,7-triazacyclononane-1,4,7-triacetic acid (NOTA); Desferrioxamine, DOTA-Tyr(3)-octreotide (DOTATOC); DOTA-Tyr(3)-Tyr(8)-octreotide (DOTATATE); DOTA-1-naphtyl-alanine (DOTANOC); DOTA-benzothienyl-alanine (DOTA-BOC); DOTA-bombesin; DOTA-arginine-glycine-aspartic acid-bombesin (DOTA-RGD-bombesin); 1,4,7-triazacyclononane-1,4,7-triacetic acid-RGD (NOTA-RGD); 3,6,9,15-tetraazabicyclo[9.3.1]pentadeca-1(15),11,13-triene-3,6,9-triacetic acid-RGD (PCTA-RGD); DOTA-albumin; DOTA-human epidermal growth factor; 1,4,7-triazacyclononane-1-[methyl(2-carboxyethyl)phosphinic acid]-4,7-bis[methyl(2-hydroxymethyl)phosphinic acid-integrin alpha(IIb)beta(3)-specific cyclic hexapeptide (NOPO-RGDfK); 1,4,7-triazacyclononane-1,4-bis(acetic acid)-7-(2-glutaric acid) (NODAGA); NOPO-NaI(3)-octreotide conjugate (NOPO-NOC); and 1,4,7-triazacyclononane-1,4,7-tris[(2-carboxyethyl) methylenephosphinic acid] (TRAP(RGD)₃).

26. The composition of claim 24, wherein the carrier molecule is an antibody or a fragment thereof.

27. The composition of claim 24, wherein the carrier molecule is a peptide or a fragment thereof.

28. The composition of claim 24, wherein the carrier molecule is a prostate-specific membrane antigen (PSMA).

29. The composition of claim 24, wherein the carrier molecule targets a human tissue.

30. The composition of claim 24, wherein the human tissue is selected from the following: thyroid, brain, gastrointestinal, pancreas, spleen, kidney, neuroendocrine tumors, renal cell carcinoma, small cell lung cancer, breast cancer, prostate cancer, and malignant lymphoma.
